(19) United States
(12) Patent Application Publication
(10) Pub. No.: US 2011/0105600 A1
(43) Pub. Date: May 5, 2011

(54) SYNERGISTIC COMPOSITION OF (-)-HYDROXYCITRIC ACID WITH MONOTERPENE AND A METHOD TO ENHANCE SATIETY

(75) Inventors: Daniel Elliot Clouatre, Santa Monica, CA (US); Dallas Lavoe Clouatre, Santa Monica, CA (US); Jeff Taylor, Greenwood, SC (US); Tyler White, Greenwood, SC (US)

(73) Assignees: Glykon Technologies Group, LLC, Santa Monica, CA (US); PFIZER INC., New York, NY (US)

(21) Appl. No.: 12/608,264

(22) Filed: Oct. 29, 2009

(51) Int. Cl. A61K 31/194; A61K 31/35; A61P 3/00

(52) U.S. Cl. 514/456; 514/574

(57) ABSTRACT

The present disclosure provides synergistic composition comprising (−)-hydroxycitric acid, its salts, amides and esters in conjunction with monoterpenes for enhancing satiety. The disclosure is also related to the use of synergistic composition in food and pharmaceutical compositions. The present disclosure helps in controlling obesity by enhancing satiety.

### TECHNICAL FIELD

The disclosure relates to synergistic compositions comprising (−)-hydroxycitric acid, its salts, amides and esters in conjunction with monoterpene for enhancing satiety.

### BACKGROUND

Obesity is a major health problem in developed and developing countries. Associated with obesity are increased risks for hypertension, diabetes Type II and dyslipidemia—components of the Metabolic Syndrome. Also, arthritis, certain cancers, chronic low-grade inflammation and serious hormonal imbalances in women have been linked with obesity, albeit whether as cause, effect or both remains controversial. (Bray G A. The epidemic of obesity and changes in food intake: the Fluoride Hypothesis. Physiol Behav. 2004 August; 82(1):115-21.) Currently-available drugs offer only modest therapeutic benefits accompanied by numerous adverse side effects. This is evidenced, in part, by the high attrition rates in clinical trials for Orlistat and Sibutramine. (Rucker D, Padwal R, Li S K, Curioni C, Lau D C, Long term pharmacotherapy for obesity and overweight: updated meta-analysis. BMJ 2007 Dec. 8; 335(7631):1194-9.) So-called “natural” or “health food” approaches have shown little success, which is reflected in an actual decline in diet product launches and the negative academic evaluation of available weight loss products in the face of increased levels of obesity. (Merrett N. Uncertainty marks new year weight control development. Nutraingredients.com 2009 Jan. 8; Heller L. BMJ editorial says only drugs help weight loss. 2008 Nov. 27.) Similarly revealing is the number of “health food” weight loss supplements officially found to contain pharmaceutical agents. (FDA Expands Warning to Consumers About Tainted Weight Loss Pills. FDA News. January 2009.)

A possible exception to the litany of diet product disappointments is (−)-hydroxycitric acid (abbreviated herein as HCA), a naturally-occurring substance found chiefly in fruits of the species of Garcinia, and several synthetic derivatives of citric acid that have been investigated extensively with regard to their ability to inhibit the production of fatty acids from carbohydrates, to suppress appetite, and to inhibit weight gain. (Sullivan C, Triscari J. Metabolic regulation as a control for lipid disorders. I. Influence of (−)-hydroxycitrate on experimentally induced obesity in the rodent. Am J Clin Nutr. 1977 May; 30(5):767-76.) Numerous other benefits have been attributed to the use of HCA, including, but not limited to an increase in the metabolism of fat stores for energy and an increase in thermogenesis. The commonly offered explanation for the effects of HCA is that this compound inhibits the actions of cytoplasmic (cytosolic) ATP:citrate lyase. (Clouatre D, Rosenbaum M E. The Diet and Health Benefits of HCA (Hydroxicitric Acid), 1994.) Weight loss benefits were attributed to HCA, its salts and its lactone in U.S. Pat. No. 3,764,692 granted to John M. Lowenstein in 1973. Lowenstein described a variety of possible pharmaceutical salts of HCA based upon alkali metals, e.g., potassium and sodium, and alkaline earth metals, e.g., calcium and magnesium.

Free (−)-hydroxycitric acid, calcium, magnesium and potassium salts of HCA and poorly characterized mixtures of two or more of these minerals, usually substantially contaminated with sodium—and, sometimes, even free chloride ion with only the sodium has been removed, currently exist on the American market. Calcium/sodium salts have been sold widely since at least as early as 1992. Most of the HCA sold to date consists of calcium salts of varying degrees of purity and, more recently, of poorly characterized calcium and potassium mixtures. For instance, the currently best selling HCA salt (potassium-calcium hydroxycitrate) typically contains ≧10 percent impurities and the product specification allows for approximately 25 percent ± variations in the mg/gram of the potassium and calcium cations. (Shara M, Ohia S E, Schmidt R E, Yasmin T, et al. Physico-chemical properties of a novel (−)-hydroxycitric acid extract and its effect on body weight, selected organ weights, hepatic lipid peroxidation and DNA fragmentation, hematology and clinical chemistry, and histopathological changes over a period of 90 days. Mol Cell Biochem. 2004 May; 260(1-2):171-86.) Safety issues have been raised with regard to the free acid and lactone forms of HCA due to their strong chelating properties and the risk of excessive loss of zinc from the body, a concern especially important to males both in puberty and in the later stages of life.

HCA was very extensively studied by Hoffman-La Roche. Animal tests to establish the appetite suppressing effects of HCA found that a single large oral dose or two divided oral doses totaling approximately one-fourth the size of the single dose resulted in a 10% or greater reduction in food consumption in experimental animals fed a high-sugar diet. (Minimum doses were 2.63 mmoles/kg once per day or 0.33 mmoles/kg twice per day either one hour before meals or four hours after, but not after the last meal of the day.) This result continued over many weeks, but in no case beyond approximately seven weeks, with the chronic ingestion of HCA. The appetite control mechanism of HCA was said to not involve any conditioned aversion to food, i.e., HCA does not alter taste, cause gastric distress or illness, etc. Rather, this control was thought to stem from the increased production of glycogen and/or stimulation of glucoreceptors in the liver, either of which results in early satiety through signals sent to the brain via the vagus nerve. It has now been demonstrated experimentally that the Roche position that HCA suppresses appetite through vagal afferents associated with the liver almost certainly is mistaken. In an animal trial in which the hepatic branch of the vagus was severed (hepatic branch vagotomy), there was no significant effect found with this surgery in comparison with controls. (Leonhardt M, Hrupka B J, Langhans W. Subdiaphragmatic vagal deafferentation fails to block the anorectic effect of hydroxycitrate. Physiol Behav. 2004 Sep. 15; 82(2-3):263-8.)

Human research has provided, at best, only weak support for the Roche satiety findings, which were based on animal trials and mostly restricted diets, for instance, based predominantly on glucose. Early satiety with meals has been found only under very limited conditions. Ingesting tablets or capsules, even when there was significant weight loss, has not led to significantly advanced satiety. For instance, although 1,200 mg HCA daily given as tablets (2×400 mg 50% material as Citrin® calcium hydroxycitrate taken 3 times daily before meals) for 12 weeks led to significant weight loss, there was no significant change in food intake. The findings were 3.7±3.1 kg active versus 2.4±2.9 kg placebo. Over a 3-month period, these results of less than a pound of additional weight loss per month are hardly impressive; however, the difference is significant. (Mattes R D, Bormann L. Effects of (−)-hydroxycitric acid on appetitive variables. Physiol Behav. 2000 Oct. 1-15; 71(1-2):87-94.)

A more recent trial that utilized a diet normal in caloric intake, but reduced in fat and employing prepared meals, enforced exercise, and visual inspection of capsule consumption found significant weight loss, but mealtime satiety was increased over a period of many weeks rather than days. (Preuss H G, Bagchi D, Bagchi M, Rao C V, Dey D K, Satyanarayana S. Effects of a natural extract of (−)-hydroxycitric acid (HCA-SX) and a combination of HCA-SX plus niacin-bound chromium and Gymnema sylvestre extract on weight loss. Diabetes Obes Metab. 2004 May; 6(3):171-80.) At the end of eight weeks, appetite was decreased by approximately 15.6% in the group consuming 2,800 mg/HCA per day in capsules and by 21.2% in those consuming this amount of HCA plus other ingredients. Placebo experienced no reduction in appetite. Obviously, a certain percentage of the change in appetite in the active arms at the end of eight weeks can be attributed to weight loss rather than satiety per se. More telling, a trial described below using the same HCA source at only 900 mg/day, but delivered differently, reduced appetite by 15-30% in a mere two weeks.

The reductions in appetite and the other findings in the Preuss study also appear to be inflated by the failure to properly blind the trail. Put simply, the placebo used in the trial, mircocrystalline cellulose, is light filler having a bulk density of between 0.2 and 0.4 g/cc, whereas a calcium-potassium HCA salt has a bulk density of between 0.7 and 0.9 g/cc. Even in opaque capsules, it immediately would have been obvious to all involved which capsules contained placebo and which contained the actives. The failure of the blinding answers many of the questions that have been raised regarding this study (somewhat misleadingly published as several papers over a two-year period) conducted entirely in India under Indian conditions. For instance, despite boxed meals and enforced/supervised exercise in previously sedentary subjects, in the placebo arm there either was no benefit or even a trend upward for LDL, triglycerides and total cholesterol, whereas HDL trended downward. Bodyweight in placebo barely budged at either 4 or 8 weeks. In effect, there was no placebo response despite major interventions, any one of which normally produces significant results in LDL, HDL, triglycerides, total cholesterol, and body weight. To take but one parameter, the failure to show weight loss in placebo under these experimental conditions is out of line with the great preponderance of published studies. (Truby H, Baic S, deLooy A, Fox K R, et al. Randomised controlled trial of four commercial weight loss programmes in the UK: initial findings from the BBC “diet trials”. BMJ. 2006 Jun. 3; 332(7553):1309-14.) Even taking the study at face value, the reductions in appetite reported were small and required many weeks to become significant.

Human trials with HCA showing the rapid onset of meal-linked satiety are limited to only one study. Current HCA products have been shown to induce meal satiety (as opposed to reducing snacking) within a reasonable time period of days rather than weeks only under the very limited condition of being dissolved in 100 ml tomato juice just prior to ingestion timed approximately an hour before lunch and supper, then two hours after the evening meal to reduce snacking. In a trial published in 2002, although food intake decreased 15-30% there was no significant weight loss over a 2-week period. (Westerterp-Plantenga M S, Kovacs E M. The effect of (−)-hydroxycitrate on energy intake and satiety in overweight humans. Int J Obes Relat Metab Disord. 2002 June; 26(6):870-2.) The researchers themselves noted the problematic nature of HCA delivery by way of the observation “Prevention of degradation and bio-availability was documented.” The satiety found in this study using much smaller amounts of HCA than in Preuss 2004 (900 mg vs. 2800 mg) not only appeared far more quickly, but was more powerful than that reported in the 2004 Indian study even at the end of the 8 week trial.

The foregoing studies are representative. The only reasonable conclusion that can be drawn from the literature available on HCA in humans is that there is little impact on meal satiety when the compound is delivered via capsules or tablets if the relevant period is counted in days rather than weeks. To date, satiety has been demonstrated in humans only when HCA is dissolved and delivered in a substantial amount of tomato juice approximately an hour before meals. Capsules and tablets have proved to be ineffective for inducing meal satiety. Indeed, despite the Indian trial described above and published as several papers in 2004 and 2005, two of the leading American researchers in the field of bariatrics in 2007 continued to express skepticism regarding the viability of HCA as a diet product. (Bray G A, Greenway F L. Pharmacological treatment of the overweight patient. Pharmacol Rev. 2007 June 59(2):151-84.)

Delivering meal satiety with HCA under the only approach shown to work, i.e., mixing in a large volume of tomato juice just before consuming 60 minutes prior to meals, is extremely onerous. The components of this approach include a) preventing degradation of the HCA, b) insuring the complete release of the HCA, and c) insuring bioavailability of the HCA. Degradation is a major issue. Not one of the proposed ready-to-drink HCA preparations or HCA “waters” that have been marketed has succeeded, in large part because of degradation. HCA, as is well established, very readily binds to gums, fibers and pectins. It also binds to many phytochemicals, such catechins and polyphenols more generally. Leaving HCA in prepared beverage preparations, especially those that have been heat pasteurized, encourages these reactions and also induces the rapid formation of the HCA lactone. The lactone is almost totally ineffective for satiety and other health purposes. (Lowenstein J M, Brunengraber H. Hydroxycitrate. Methods Enzymol. 1981; 72:486-97.) However, the lactone does have at least one negative impact—it has a type of toxicity, probably due to its binding zinc and leading to its excretion from the body. The lactone is labile to the acid, so the chelation most likely is due to the free acid. (Burdock G, Soni M, Bagchi M, Bagchi D. Garcinia cambogia toxicity is misleading. Food Chem Toxicol. 2005 November; 43(11):1683-4; author reply 1685-6. Erratum in: Food Chem Toxicol. 2007 March; 45(3):515.) Studies by other researchers utilizing fully reacted HCA salts have found no toxicities.

The other two issues of complete release and bioavailability, similarly, pose daunting challenges. Westerterp-Plantenga and Kovacs chose tomato juice as a vehicle precisely because its pH would insure that the HCA salt was fully dissolved. By using a relatively high volume vehicle, they also insured that the dissolved salt would be exposed to any possible receptors in the stomach and intestine as well as allowing for better uptake. In this sense, their approach replicates the usually successful delivery of HCA by gavage. It is not accidental that no one has been able to duplicate Westerterp-Plantenga and Kovacs' results by means of other forms of delivery until now.

To be sure, others have proposed orange oil and its active component d-limonene as an anorectic agent. (Brudnak M A. Weight-loss drugs and supplements: are there safer alternatives. Med Hypotheses 2002; 58:28-33.) Evidence for anorectic effects of monoterpenes, such as d-limonene, has been available for many years. (Von Burg R. Limonene. J Appl Toxicol. 1995 November-December; 15(6):495-9.) However, the oral dosages required as seen in animal models have been very substantial. Moreover, animal models and humans often differ dramatically in the details. In the case of d-limonene, an oral dose of 100 mg/kg, i.e., a bolus dose of 7 grams for a 70 kg human, produced mild satiety for 10 hours as well as slight fatigue and eructation for four hours. (Crowell P L, Elson C E, Bailey H H, et al. Human metabolism of the experimental cancer therapeutic agent d-limonene. Cancer Chemother Pharmacol 1994; 35:31-37.) The satiety is desirable, whereas the fatigue and eructation are not. Moreover, an effective dosage of 7 grams required as a bolus to elicit merely mild satiety is not acceptable. It should be noted that d-limonene has GRAS status in the US and is deemed safe in humans based on clinical data. d-Limonene has been found to be safe and without gradable toxicity when 100 mg/kg (equivalent to about 7 g bolus for an average adult male) was ingested. (Sun J. D-Limonene: safety and clinical applications. Ahern Med Rev. 2007 September; 12(3):259-64.)

From the foregoing, it can be gleaned that neither HCA salts nor d-limonene have proven to be effective satiety enhancers, and certainly not at the dosage levels or under the conditions that lend themselves to supporting weight loss via normal means of intake. Except with one very special and, in practice, quite onerous delivery system, HCA salts have failed to produce significant satiety over the short term (days as opposed to weeks) in trials. d-Limonene has been demonstrated to induce satiety only at an unacceptably high level of intake that leads to fatigue and eructation.

In an effort to overcome limitations, the Inventors have a synergistic composition involving the compound d-limonene in conjunction with HCA.

### SUMMARY

The present disclosure is in relation to a synergistic composition comprising (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

In another embodiment of the present disclosure, further comprises excipients in a range of about 2%[Wt/Wt] to about 20%[Wt/Wt], preferably about 15%[Wt/Wt].

In still another embodiment of the present disclosure, the (−)-hydroxycitric acid is selected from but not limited to a group comprising its salt, amide and ester, preferably the salts.

In yet another embodiment of the present disclosure, the salt of (−)-hydroxycitric acid is selected from but not limited to a group comprising salts of potassium, sodium, magnesium, calcium, potassium-magnesium and potassium-calcium, preferably potassium-magnesium salt.

In yet another embodiment of the present disclosure, the monoterpene is selected from but not limited to a group comprising d-limonene, perillyl alcohol, carveol, carvone, cineole, geraniol, nerol and perilla aldehyde.

In yet another embodiment of the present disclosure, excipients are selected from but not limited to group a comprising lecithin, beeswax, glycerol monostearate, fumed silicon dioxide and other surface-tension increasing excipients.

The present disclosure is also in relation to a method of preparation of synergistic composition comprising (−)-hydroxycitric acid in range of about 40%[Wt/Wt] to about 60%[Wt/Wt], preferably about 50%[Wt/Wt], monoterpene in range of about 20%[Wt/Wt] to about 50%[Wt/Wt], preferably about 35%[Wt/Wt], and excipients in range of about 2%[Wt/Wt] to about 20%[Wt/Wt], preferably about 15%[Wt/Wt], said method comprising steps of:
a. mixing the monoterpene and the excipient(s) to form first mixture;
b. mixing (−)-hydroxycitric acid with the first mixture to form a second mixture;
c. adding excipient(s) to the second mixture to obtain a third mixture; and
d. heating the third mixture to obtain the synergistic composition.

In yet another embodiment of the present disclosure, the mixing of monoterpene with excipient(s) is for a period ranging from about 3 minutes to a period of about 8 minutes, preferably for about 5 minutes.

In yet another embodiment of the present disclosure, the first mixture is stirred at a speed ranging from about 150 RPM to a speed ranging about 250 RPM, preferably about 200 RPM.

In yet another embodiment of the present disclosure, the excipient for step (a) is selected from but not limited to a group comprising lecithin, beeswax, glycerol monostearate and fumed silicon dioxide preferably lecithin

In yet another embodiment of the present disclosure, the second mixture is stirred at about a speed ranging from about 4000 RPM to a speed ranging about 6000 RPM, preferably about 5000 RPM.

In yet another embodiment of the present disclosure, the excipient for step (c) is selected from but not limited to a group comprising lecithin, beeswax, glycerol monostearate and fumed silicon dioxide, preferably beeswax.

In yet another embodiment of the present disclosure, the heating of the third mixture is carried out at a temperature ranging from about 60° C. to a temperature of about 80° C., preferably about 70° C.

In yet another embodiment of the present disclosure, the heating is for a period ranging from about 20 minutes to a period of about 40 minutes, preferably about 30 minutes.

In yet another embodiment of the present disclosure, the third mixture is stirred at about a speed ranging from about 150 RPM to a speed ranging about 250 RPM, preferably about 200 RPM.

In yet another embodiment of the present disclosure, further comprises of cooling the heated mixture to a temperature ranging from about 25° C. to a temperature of about 35° C., preferably about 30° C.

The present disclosure is also in relation to a method of preparation of synergistic composition comprising (−)-hydroxycitric acid in range of about 40%[Wt/Wt] to about 60%[Wt/Wt], preferably about 50%[Wt/Wt], monoterpene in range of about 20%[Wt/Wt] to about 50%[Wt/Wt], preferably about 35%[Wt/Wt], said method comprising addition of (−)-hydroxycitric acid to a solution comprising a monoterpene.

The present disclosure is also in relation to a method for enhancing satiety in a subject in need thereof comprising administering an effective amount of synergistic composition comprising (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

In yet another embodiment of the present disclosure, the synergistic composition further comprises excipients in a range of about 2% [Wt/Wt] to about 20%[Wt/Wt], preferably about 15%[Wt/Wt].

In yet another embodiment of the present disclosure, the excipients are selected from but not limited to group comprising lecithin, beeswax, glycerol monostearate and fumed silicon dioxide and other surface-tension increasing excipients.

In yet another embodiment of the present disclosure, the subject is selected from but not limited to a group comprising human being, animal and bird.

In yet another embodiment of the present disclosure, the synergistic composition is in a form selected from but not limited to a group comprising tablet, capsule, suspension, powder and particles.

In yet another embodiment of the present disclosure, the synergistic composition is used in a form selected from a group comprising but not limited to food product, confectionery, beverage and drug.

The present disclosure is also in relation to a confectionery comprising synergistic composition, wherein said synergistic composition comprises (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

In yet another embodiment of the present disclosure, said synergistic composition comprises excipients in a range of about 2% [Wt/Wt] to about 20%[Wt/Wt], preferably about 15%[Wt/Wt].

The present disclosure is also in relation to a food product comprising synergistic composition, wherein said synergistic composition comprises (−)-hydroxycitric acid in a range of about 40%[Wt/Wt] to about 60%[Wt/Wt], preferably about 50%[Wt/Wt], monoterpene in a range of about 20%[Wt/Wt] to about 50%[Wt/Wt], preferably about 35%[Wt/Wt].

In yet another embodiment of the present disclosure, said synergistic composition comprises excipients in a range of about 2% [Wt/Wt] to about 20%[Wt/Wt], preferably about 15%[Wt/Wt].

The present disclosure is also in relation to a drug comprising synergistic composition, wherein said synergistic composition comprises (−)-hydroxycitric acid in a range of about 40%[Wt/Wt] to about 60%[Wt/Wt], preferably about 50%[Wt/Wt], monoterpene in a range of about 20%[Wt/Wt] to about 50%[Wt/Wt], preferably about 35%[Wt/Wt].

In yet another embodiment of the present disclosure, said synergistic composition comprising excipients in a range of about 2% [Wt/Wt] to about 20%[Wt/Wt], preferably about 15%[Wt/Wt].

The present disclosure is also in relation to a beverage comprising of synergistic composition, wherein said synergistic composition comprises (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

The inventors have found that food and pharmaceutical compositions containing (−)-hydroxycitric acid, its salts, amides and esters can be employed in conjunction with monoterpenes to enhance meal-related satiety beyond that found with either item alone and beyond a merely additive effect. Previously, HCA meal-related satiety in humans has been demonstrated only under one very special condition described above and has failed to appear when HCA was delivered in capsules, tablets, bottled waters, and so forth and so on. d-Limonene, when ingested, has been found to induce satiety in humans only when consumed at the rate of 100 mg/kg, i.e., a bolus dose of 7 grams for a 70 kg human, and this dose led to side effects. Similar dosages and effects have been found with perillyl alcohol.

In this combination, HCA delivered in the form of its potassium or potassium-magnesium salt is efficacious at singly delivered dosages of between 1 and 5 grams, preferably at a dosage of between 2 and 3 grams for most individuals. Other salts, amides and esters are active at individual dosage ranges, with, for instance, the sodium salt acting similarly to the potassium salt whereas salts containing calcium are less active. d-Limonene requirements in conjunction with HCA salts at singly delivered dosages run from 0.5 to 5 grams, with the range suitable for most purposes and individuals being from 1 to 3 grams. The dosage preferably should be given twice daily 30-60 minutes prior to meals with other regimens for special purposes and circumstances.

### DETAILED DESCRIPTION

As stated earlier to overcome the limitations with respect to HCA and d-limonene, the Inventors have invented a synergistic composition involving the compound d-limonene in conjunction with HCA. The quite surprising disclosure by the Inventors is that HCA and d-limonene can be combined to deliver powerful satiety often even with the initial dose and routinely within one to two days of treatment. At first, two of the inventors experimented taking HCA and d-limonene made up separately. Then, as reported in Example 1 below, in a small pilot trial 5 of 6 subjects reported satiety ranging from mild to extremely robust and onset beginning either with the initial dose or within the first 2 days. Moreover, the required amount of HCA salt (approximately 50% HCA) to achieve this level of satiety is only on the order of 2 grams per serving and the amount of d-limonene is only on the order of 1 gram per serving with two servings per day 60 minutes prior to lunch and supper being recommended. This amount has been found efficacious in individuals weighing more than 250 pounds. In lighter individuals, smaller dosages may prove effective. This combination can be delivered via drinks and specially prepared confections. It, furthermore, has the striking utility of enhancing satiety even when delivered via liquid-filled hard shell capsules, whereas no previous capsule or tablet HCA delivery system has proven successful in enhancing meal satiety. In asmuch as the amount of d-limonene required is low, there are no side effects, such as fatigue and eructation, as are found with high intakes of the monoterpene.

Taking d-limonene as a representative monoterpene (any of a class of terpenes C₁₀H₁₆ containing two isoprene units per molecule), selected other members of the class, for instance, perillyl alcohol can be utilized similarly to enhance meal satiety in conjunction with HCA. Perillyl alcohol in humans demonstrates a safety profile and anorectic impact similar to d-limonene, but its anti-cancer properties are much more pronounced. (Ripple G H, Gould M N, Arzoomanian R Z, Alberti D, et al. Phase I clinical and pharmacokinetic study of perillyl alcohol administered four times a day. Clin Cancer Res. 2000 February; 6(2):390-6 and also Belanger J T. Perillyl alcohol: applications in oncology. Ahern Med Rev. 1998 December; 3(6):448-57.) Depending on the trial, the maximum tolerated dose for d-limonene is approximately 8 g/m² per day and that for perillyl alcohol is 8400 mg/m² per day (Dosage related to total body surface area.). Consumption of d-limonene leads to the appearance of perillyl alcohol in the blood of subjects. Researchers have found that 40 oz of Mediterranean-style lemonade contained 596 mg d-limonene and that this led to maximal blood concentrations of perillyl alcohol within approximately one hour under experimental conditions. (Chow H H, Salazar D, Hakim I A. Pharmacokinetics of perillic acid in humans after a single dose administration of a citrus preparation rich in d-limonene content. Cancer Epidemiol Biomarkers Prey. 2002 November 11(11):1472-6.) Some other ingestible monoterpenes that might be utilized for the Disclosure, dosage requiring individual titration and possibly mixed for delivery together, include carveol (in spearmint oil), carvone (in caraway seed and spearmint oil), perilla aldehyde (in the annual herb perilla) and geraniol (in lemongrass oil).

Various salts of (−)-hydroxycitric acid (calcium, magnesium, potassium, sodium and mixtures of these) have been available commercially for several years. Any of these materials can be used to fulfill the disclosure revealed here, but with varying degrees of success. These materials are generally useful in this descending order of efficacy: potassium salt, potassium-magnesium salt, sodium salt, potassium-calcium salt, magnesium salt and the calcium salt. The previously patented hydroxycitric acid derivatives (mostly amides and esters of hydroxycitric acid, the patents for which are now expired) likely are roughly equivalent to the HCA sodium salt in efficacy. The pure potassium and sodium salts, in practice, are less desirable either due to the sodium content or to major difficulties in handling because of their hygroscopic nature.

d-Limonene, perillyl alcohol and related monoterpenes should be in their natural chemical forms and of food grade or better quality. Citrus-derived monoterpenes must be carefully screened to avoid pesticide residue. Some other ingestible monoterpenes that might be utilized for the disclosure, dosage requiring individual titration and possibly mixed for delivery together, include carveol (in spearmint oil), carvone (in caraway seed and spearmint oil), perilla aldehyde (in the annual herb perilla), geraniol (in lemongrass oil), cineole and nerol

Methods for taking advantage of the present disclosure include, but are not limited to employment in specially prepared foodstuffs, specially delivered drinks, and liquid-filled capsules. With regard to the latter, it is far easier to produce a stable liquid-filled hard shell capsule than to produce a soft gelatin capsule. Among other things, the hygroscopic nature of the more active HCA salts makes the shelf life of soft gelatin capsules highly problematic. Employment of less hygroscopic salts utilizing calcium or the so-called di- and tri-metal salts, all of which are less active and natively less well absorbed, as can be shown in pharmacokinetic studies, leads to a trade-off of stability for inclusion of much larger amounts of the salts in question.

In the Examples, Capsugel hard shell capsules have been showcased because of the low starting moisture content in the capsule and resistance to degradation due to hygroscopic contents. Those skilled in the art may overcome these issues using soft gelatin formulations, but at the considerable expense of lower potencies, shorter shelf life and the requirement of more stabilizers. In the Examples below, another issue is that of separation. This issue is resolved by various means. When it remains a potential issue, this is remarked.

Examples of preparation of formulation are given below for understanding, it should not be construed that it limits the scope of the disclosure for those examples.

### Example 1

### A Low-Limonene Capsule Formulation and Pilot Trial

| | | Amount | |
|---|---|---|---|
| Item # | Ingredient | (mg/capsule) | Percent |
| 1 | Potassium-Magnesium | 750 | 54.95% |
| | HCA Salt | | |
| 2 | D-Limonene Food | 350 | 25.64% |
| | Grade | | |
| 3 | Lecithin | 185 | 13.55% |
| 4 | Beeswax | 80 | 5.86% |
| | Total | 1365 | 100% |

The method of preparation is as follows:

Items 2 and 3 are added together and stirred for a minimum of 5 minutes using a 3-Blade mixer at 200 RPM to achieve homogeneity. Then, item 1 is slowly added to the mixture. This is stirred constantly while adding the powder, adjusting the stir speed to maintain a gentle vortex. Once item 1 is completely added, this dispersion is sheer mixed at 5000 RPM using the Silverson L4RT for 5 minutes, or until there are no longer any visible clumps. At this point, item 4 is added and the mixture is heated to 70° C. This is stirred at 200 RPM (adjusted speed to maintain vortex) while heating. Once the temperature reached 70° C., the mixture continued to be stirred while maintaining this temperature for approximately 30 minutes or until all the beeswax melted. Then, while gently stirring, the mixture is slowly cooled down to around 30° C. to dose into capsules. Capsules are filled to within 2 mm from the top cut edge of the size 000 capsules and sealed using the LEMS process.

This preparation is tested with 6 subjects taking 3 capsules twice per day one hour before lunch and supper. Other than initially noticing a slight odor of orange oil, which passed after the first day's use, there are no side effects. Two subjects experienced early satiety the very first meal after the initial dose and the satiety early in meals continued for the five days for which capsules were supplied. One subject experienced early satiety on the second day and described the effect as, on a scale of 1 to 10, a “9.” The satiety effect continued for the two weeks for which this subject had access to capsules. Two subjects reported mild satiety beginning the second day of use. The last subject did not report any increase in meal satiety during the five days of the trial. Hence, 5 of 6 subjects reported satiety ranging from mild to extremely robust and onset beginning either with the initial dose or within the first 2 days.

### Example 2

### A Low-Limonene High HCA Capsule Formulation

| | | Amount | |
|---|---|---|---|
| Item # | Ingredient | (mg/capsule) | Percent |
| 1 | Potassium-Magnesium | 800 | 58.18 % |
| | HCA Salt | | |
| 2 | D-Limonene Food | 350 | 25.45% |
| | Grade | | |
| 3 | Lecithin | 150 | 10.91% |
| 4 | Beeswax | 75 | 5.45% |
| | Total | 1375 | 100% |

The method of preparation is as follows:

Items 2 and 3 are added together and stirred for a minimum of 5 minutes using a 3-Blade mixer at 200 RPM to achieve homogeneity. Then, item 1 is slowly added to the mixture. This is stirred constantly while adding the powder, adjusting the stir speed to maintain a gentle vortex. Once item 1 is completely added, this dispersion is sheer mixed at 5000 RPM using the Silverson L4RT for 5 minutes, or until there are no longer any visible clumps. At this point, item 4 is added and the mixture is heated to 70° C. This is stirred at 200 RPM (adjusted speed to maintain vortex) while heating. Once the temperature reached 70° C., the mixture continued to be stirred while maintaining this temperature for approximately 30 minutes or until all the beeswax melted. Then, while gently stirring, the mixture is slowly cooled down to around 30° C. to dose into capsules. Capsules are filled to within 2 mm from the top cut edge of the size 000 capsules and sealed using the LEMS process.

### Example 3

### A High-Limonene Capsule Formulation

| | | Amount | |
|---|---|---|---|
| Item # | Ingredient | (mg/capsule) | Percent |
| 1 | Potassium-Magnesium | 750 | 48.39% |
| | HCA Salt | | |
| 2 | D-Limonene Food Grade | 750 | 48.39% |
| 3 | Lecithin | 25 | 1.61% |
| 4 | Beeswax | 25 | 1.61% |
| | Total | 1550 | 100% |

The method of preparation is as follows:

Items 2 and 3 are added together and stirred for a minimum of 5 minutes using a 3-Blade mixer at 200 RPM to achieve homogeneity. Then, item 1 is slowly added to the mixture. This is stirred constantly while adding the powder, adjusting the stir speed to maintain a gentle vortex. Once item 1 is completely added, this dispersion is sheer mixed at 5000 RPM using the Silverson L4RT for 5 minutes, or until there are no longer any visible clumps. At this point, item 4 is added and the mixture is heated to 70° C. This is stirred at 200 RPM (adjusted speed to maintain vortex) while heating. Once the temperature reached 70° C., the mixture continued to be stirred while maintaining this temperature for approximately 30 minutes or until all the beeswax melted. Then, while gently stirring, the mixture is slowly cooled down to room temperature. This resulted in a formulation that is thin with separation presenting an issue.

### Example 4

### A Medium-Limonene Capsule Formulation

| | | Amount | |
|---|---|---|---|
| Item # | Ingredient | (mg/capsule) | Percent |
| 1 | Potassium-Magnesium | 750 | 51.1% |
| | HCA Salt | | |
| 2 | D-Limonene Food | 400 | 34.1% |
| | Grade | | |
| 3 | Lecithin | 250 | 9.88% |
| 4 | Glycerol Monostearate | 10 | 1.50% |
| 5 | Aerosil 380 Silicone | 40 | 3.41% |
| | Dioxide | | |
| | Total | 1467.5 | 100% |

The method of preparation is as follows:

Items 2 and 3 are added together and stirred for a minimum of 5 minutes using a 3-Blade mixer at 200 RPM to achieve homogeneity. Then, item 1 is slowly added to the mixture. This is stirred constantly while adding the powder, adjusting the stir speed to maintain a gentle vortex. Once item 1 is completely added, this dispersion is sheer mixed at 5000 RPM using the Silverson L4RT for 5 minutes, or until there are no longer any visible clumps. At this point, item 4 is added and the mixture is heated to 70° C. This is stirred at 200 RPM (adjusted speed to maintain vortex) while heating. Once the temperature reached 70° C., the mixture continued to be stirred while maintaining this temperature for approximately 30 minutes or until all the glycerol monostearate had melted. Then, while gently stirring, the mixture is slowly cooled down to room temperature. Then, item 5 is slowly added to the mixture. This is stirred into the formula as it is being added. This resulted in an increased viscosity without separation occurring over time.

### Example 5

### A Medium-Limonene Capsule Formulation

| | | Amount | |
|---|---|---|---|
| Item # | Ingredient | (mg/capsule) | Percent |
| 1 | Potassium-Magnesium HCA | 750 | 51.1% |
| | Salt | | |
| 2 | D-Limonene Food Grade | 500 | 34.1% |
| 3 | Lecithin | 145 | 9.88% |
| 4 | Glycerol Monostearate | 22.5 | 1.50% |
| 5 | Aerosil 380 Silicone Dioxide | 50 | 3.41% |
| | Total | 1467.5 | 100% |

The method of preparation is as follows:

Items 2 and 3 are added together and stirred for a minimum of 5 minutes using a 3-Blade mixer at 200 RPM to achieve homogeneity. Then, item 1 is slowly added to the mixture. This is stirred constantly while adding the powder, adjusting the stir speed to maintain a gentle vortex. Once item 1 is completely added, this dispersion is sheer mixed at 5000 RPM using the Silverson L4RT for 5 minutes, or until there are no longer any visible clumps. At this point, item 4 is added and the mixture is heated to 70° C. This is stirred at 200 RPM (adjusted speed to maintain vortex) while heating. Once the temperature reached 70° C., the mixture continued to be stirred while maintaining this temperature for approximately 30 minutes, or until all the glycerol monostearate has melted. Then, while gently stirring, the mixture is slowly cooled down to room temperature. Then, item 5 is slowly added to the mixture. This is stirred into the formula as it is being added. This resulted in an increased viscosity, but separation is still present over time, indicating a short shelf life.

### Example 6

### Perillyl Alcohol Plus HCA Capsule Formulations

Perillyl alcohol displays solvent and anorectic characteristics similar to those of d-limonene. However, inasmuch as the carrying capacity/surface tension is slightly different, the preceding formulas need to be revised in each instance to take into account these differences, which primarily affect capsule filling and separation.

### Example 7

### Multiple Monoterpene Formulations

The monoterpenes d-limonene and perillyl alcohol are particularly suitable to the Invention because they are relatively inexpensive and nontoxic even at extremely elevated levels of intake. d-Limonene is the primary component of orange oil. However, a number of other monoterpenes exist that can be incorporated into capsule, food and drink deliveries. Most of these other monoterpenes presently are used widely for flavoring and fragrance purposes. Some bring particular health benefits of their own. Due to their greater expense and their relatively strong flavors and/or fragrances, they typically would not be used as the sole or even the primary monoterpene sources for the Invention, but rather would be employed to impart desired taste and fragrance characteristics, particularly to food and drink preparations. An extended list of monoterpenes would include at the very least carveol, carvone, cineole, geraniol, nerol and perilla aldehyde. Monoterpenes such as nerol and geraniol are Generally Regarded As Safe (GRAS). Carveol typically is incorporated at levels up to 50 ppm for flavoring; cineole is incorporated at levels up to 190 ppm for flavoring; and perilla aldehyde would not be incorporated at a level above 100 ppm. As an example of the flavoring effects of monoterpenes, nerol/geraniol blends are harnessed to give sweet fruity taste characteristics with citrus and raspberry overtones when incorporated at the rate of 10 ppm by flavor masters. Other flavors that commonly are accentuated through the proper mixing of monoterpenes are rose, citrus, caraway and mint. Safety profiles are good on these flavor/fragrance monoterpenes. For instance, the acute oral LD50 for geraniol is 3,600 mg/kg body weight in the rat and the oral LD50 for nerol is 4,500 mg/kg body weight in the rat.

### Example 7

### A Confectionery Formulation

The HCA plus d-limonene combination readily can be incorporated into various confections based on centers or cores. Experimentally, the combination is easily contained in gelatin-based centers including sugars and alcohol sugars such as sucrose, maltose, trehalose, isomaltose, sorbitol, xylitol and so forth. (Gums, pectins and similar binders, however, are to be avoided.) HCA has been shown to dramatically slow the crossing of glucose from the gut into the blood, hence there is little fear of inducing blood sugar spikes with these combinations. The primary concerns are two: HCA is hygroscopic, whereas the monoterpenes d-limonene and perillyl alcohol act as solvents. Hence the core must be surrounded by a barrier against moisture, but that barrier should not consist of items that dissolve easily in monoterpenes. The solution is initially to apply coatings of sugar or polyols to the core by means known to those skilled in the art. Subsequently, a second coating of a fat-based or waxy edible, such as chocolate (or variations such as chocolate and tea extracts), is applied to complete the process with a moisture barrier. The range of centers possible is limited only by the skill and art of the flavor master. The sole requirement in terms of efficacy is that the resulting product supply an efficacious HCA salt preferably in the range of 2-3 grams and a monoterpene in the range of 1 to 3 grams per serving. The broader range given under “Summary” takes into account extremes in body weight and other varying circumstances sometimes encountered.

### Example 8

### A Drink Formulation Utilizing Separation of Components

As a flavoring, d-limonene commonly is used in various drinks and foodstuffs. It is particularly appropriate for orange and citrus-flavored drinks and for milk-based drinks with “orange Julius” and chocolate themes. Hence, it is possible in producing such beverages to incorporate a monoterpene with the range of 1 to 3 grams per serving into formulations. The difficulty lies in incorporating 2-3 grams of an appropriate HCA salt. However, this problem can be overcome through the use of any one of a number of approaches for packaging a dry preparation above a liquid preparation, including liquids that have been pasteurized. A more mundane approach for accomplishing the same end is to include the HCA powder in stick pack delivery attached to the prepared beverage containing the monoterpene.

A simple recipe for producing an orange-flavored syrup for beverages is this:

| Orange flavor | 50 | g |
|---|---|---|
| Phosphoric acid | 15 g (80 wt. % aq. soln.) | |
| Malic acid | 2.5 | g |
| Citric acid | 1.5 | g |
| Sodium chloride | 11 | g |
| Calcium chloride | 1.6 | g |
| Monopotassium phosphate | 8 | g |
| Ascorbic acid | 7 | g |
| HFCS 55 (77.degree. Brix) | 2700 | g |

| | |
|---|---|
| Purified water | sufficient to bring the total to 5000 ml |

The above syrup is adjusted for pH, titrated acidity, cloudiness, etc. to accommodate the addition of 1.5 to 2.5 grams d-limonene per 10 ounce serving when the syrup is diluted with four volumes purified water. Flavor may be boosted through the addition of the monoterpenes nerol and/or geraniol as desired for fragrance. Should be bottled in glass to avoid loss of d-limonene to absorption into plastics. Attached to each bottle is a stick pack containing 2 grams HCA salt to be added and dissolved prior to consumption.

### Example 9

### A Drink Formulation with Unitary Delivery

Advanced packaging technologies now exists that permit sterilization of all ingredients prior to packaging. There also are pressure-based sterilization systems available that perform a similar function without heating. These approaches eliminate the need for many drinks to be acidic in nature. Accordingly, utilization of these or similar technologies potentially permits the incorporation of HCA salts directly into beverage formulations along with the d-limonene. This is particularly true of milk-based formulations. HCA salts disassociate at low pH values, but at the mildly alkaline pH typical of many milk-based beverages they will remain stable if heat sterilization can be avoided and sterilization accomplished by other means. Hence those skilled in the art can take the suggestions of Example 9 and produce a drink formulation incorporating both a monoterpene component and an HCA salt component delivered as a ready to drink beverage.

We claim:

1. A synergistic composition comprising (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

2. The synergistic composition as claimed in claim 1 further comprises excipients in a range of about 2%[Wt/Wt] to about 20%[Wt/Wt], preferably about 15%[Wt/Wt].

3. The synergistic composition as claimed in claim 1, wherein the (−)-hydroxycitric acid is selected from but not limited to a group comprising its salt, amide and ester, preferably the salts.

4. The synergistic composition as claimed in claim 3, wherein the salt of (−)-hydroxycitric acid is selected from but not limited to a group comprising salts of potassium, sodium, magnesium, calcium, potassium-magnesium and potassium-calcium, preferably potassium-magnesium salt.

5. The synergistic composition as claimed in claim 1, wherein the monoterpene is selected from but not limited to a group comprising d-limonene, perillyl alcohol, carveol, carvone, cineole, geraniol, nerol and perilla aldehyde.

6. The synergistic composition as claimed in claim 2, wherein excipients are selected from but not limited to group a comprising lecithin, beeswax, glycerol monostearate, fumed silicon dioxide and other surface-tension increasing excipients.

7. A method of preparation of synergistic composition comprising (−)-hydroxycitric acid in range of about 40%[Wt/Wt] to about 60%[Wt/Wt], preferably about 50%[Wt/Wt], monoterpene in range of about 20%[Wt/Wt] to about 50%[Wt/Wt], preferably about 35%[Wt/Wt], and excipients in range of about 2%[Wt/Wt] to about 20%[Wt/Wt], preferably about 15%[Wt/Wt], said method comprising steps of:
a. mixing the monoterpene and the excipient(s) to form first mixture;
b. mixing (−)-hydroxycitric acid with the first mixture to form a second mixture;
c. adding excipient(s) to the second mixture to obtain a third mixture; and
d. heating the third mixture to obtain the synergistic composition.

8. The method as claimed in claim 7, wherein the mixing of monoterpene with excipient(s) is for a period ranging from about 3 minutes to a period of about 8 minutes, preferably for about 5 minutes.

9. The method as claimed in claim 7, wherein the first mixture is stirred at a speed ranging from about 150 RPM to a speed ranging about 250 RPM, preferably about 200 RPM.

10. The method as claimed in claim 7, wherein the excipient for step (a) is selected from but not limited to a group comprising lecithin, beeswax, glycerol monostearate and fumed silicon dioxide, preferably lecithin.

11. The method as claimed in claim 7, wherein the second mixture is stirred at about a speed ranging from about 4000 RPM to a speed ranging about 6000 RPM, preferably about 5000 RPM.

12. The method as claimed in claim 7, wherein the excipient for step (c) is selected from but not limited to a group comprising lecithin, beeswax, glycerol monostearate and fumed silicon dioxide, preferably beeswax.

13. The method as claimed in claim 7, wherein the heating of the third mixture is carried out at a temperature ranging from about 60° C. to a temperature of about 80° C., preferably about 70° C.

14. The method as claimed in claim 7, wherein the heating is for a period ranging from about 20 minutes to a period of about 40 minutes, preferably about 30 minutes.

15. The method as claimed in claim 7, wherein the third mixture is stirred at about a speed ranging from about 150 RPM to a speed ranging about 250 RPM, preferably about 200 RPM.

16. The method as claimed in claim 7 further comprising of cooling the heated mixture to a temperature ranging from about 25° C. to a temperature of about 35° C., preferably about 30° C.

17. A method of preparation of synergistic composition comprising (−)-hydroxycitric acid in range of about 40%[Wt/Wt] to about 60%[Wt/Wt], preferably about 50%[Wt/Wt], monoterpene in range of about 20%[Wt/Wt] to about 50%[Wt/Wt], preferably about 35%[Wt/Wt], said method comprising addition of (−)-hydroxycitric acid to a solution comprising a monoterpene.

18. A method for enhancing satiety in a subject in need thereof comprising administering an effective amount of synergistic composition comprising (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

19. The method for enhancing satiety as claimed in claim 18, wherein the synergistic composition further comprises excipients in a range of about 2% [Wt/Wt] to about 20%[Wt/Wt], preferably about 15%[Wt/Wt].

20. The method for enhancing satiety as claimed in claim 19, wherein the excipients are selected from but not limited to group comprising lecithin, beeswax, glycerol monostearate and fumed silicon dioxide and other surface-tension increasing excipients.

21. The method as claimed in claim 18, wherein the subject is selected from but not limited to a group comprising human being, animal and bird.

22. The method as claimed in claim 18, wherein the synergistic composition is in a form selected from but not limited to a group comprising tablet, capsule, suspension, powder and particles.

23. The method as claimed in claim 18, wherein the synergistic composition is used in a form selected from a group comprising but not limited to food product, confectionery, beverage and drug.

24. A confectionery comprising synergistic composition, wherein said synergistic composition comprises (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].

25. The confectionery comprising synergistic composition as claimed in claim 24, wherein said synergistic composition comprises excipients in a range of about 2% [Wt/Wt] to about 20%[Wt/Wt], preferably about 15%[Wt/Wt].

26. A food product comprising synergistic composition, wherein said synergistic composition comprises (−)-hydroxycitric acid in a range of about 40%[Wt/Wt] to about 60%[Wt/Wt], preferably about 50%[Wt/Wt], monoterpene in a range of about 20%[Wt/Wt] to about 50%[Wt/Wt], preferably about 35%[Wt/Wt].

27. The food product comprising synergistic composition as claimed in claim 26, wherein said synergistic composition comprises excipients in a range of about 2% [Wt/Wt] to about 20%[Wt/Wt], preferably about 15%[Wt/Wt].

28. A drug comprising synergistic composition, wherein said synergistic composition comprises (−)-hydroxycitric acid in a range of about 40%[Wt/Wt] to about 60%[Wt/Wt], preferably about 50%[Wt/Wt], monoterpene in a range of about 20%[Wt/Wt] to about 50%[Wt/Wt], preferably about 35%[Wt/Wt].

29. The drug comprising synergistic composition as claimed in claim 28, wherein said synergistic composition comprising excipients in a range of about 2% [Wt/Wt] to about 20%[Wt/Wt], preferably about 15%[Wt/Wt].

30. A beverage comprising of synergistic composition, wherein said synergistic composition comprises (−)-hydroxycitric acid in a range of about 40%[Wt/Wt] to about 60%[Wt/Wt], preferably about 50%[Wt/Wt] and monoterpene in a range of about 20%[Wt/Wt] to about 50%[Wt/Wt], preferably about 35%[Wt/Wt].
